(19) United States
(12) Patent Application Publication
(10) Pub. No.: US 2023/0404530 A1
(43) Pub. Date: Dec. 21, 2023

(54) PELVIC FLOOR DIAGNOSTIC-THERAPEUTIC TREATMENT CHAIR

(71) Applicant: NINA MEDICAL LTD., Nazareth (IL)

(72) Inventors: Shmuel BEN-EZRA, Pardes-Hanna (IL); Yoni HERTZBERG, Shoham (IL); Alexander VOLOVICK, Rishon LeZion (IL)

(21) Appl. No.: 18/034,873

(22) PCT Filed: Nov. 3, 2021
(86) PCT No.: PCT/IL2021/051301

§ 371 Date: May 1, 2023

Related U.S. Application Data
(60) Provisional application No. 63/109,091, filed on Nov. 3, 2020.

(51) Int. Cl. A61B 8/00; A61B 8/08; A61N 7/02

(52) U.S. Cl. CPC A61B 8/429; A61B 8/40; A61B 8/4281; A61B 8/4461; A61B 8/5207; A61N 7/02

(57) ABSTRACT

A treatment head (20, 20′) includes an ultrasound imaging probe (22) which generates a sonogram (24) of internal anatomy of a subject (25), and a HIFU transducer 26 which emits HIFU energy (31) into the subject. A chair (28) includes a chair-frame (30) and a seat (32). When the subject is sitting in the chair, a perineum (34) of the subject is acoustically coupled to the treatment head. Control circuitry (36) (a) controls movement of the treatment head relative to the seat, (b) operates the ultrasound imaging probe to generate at least one sonogram of internal anatomy of the subject through the perineum of the subject, and (c) operates the HIFU transducer to emit HIFU energy into the body of the subject through the perineum of the subject. Other embodiments are also described.

### CROSS REFERENCE TO RELATED APPLICATION

The present application claims the priority of U.S. 63/109,091 to Ben Ezra et al., filed Nov. 3, 2020, entitled, “Pelvic floor diagnostic-therapeutic treatment chair,” which is incorporated herein by reference.

### FIELD OF THE INVENTION

Applications of the present invention relate to diagnostic and therapeutic ultrasound. More specifically, applications of the present invention relate to pelvic floor diagnosis and therapy.

### BACKGROUND

Ultrasound is often used for diagnosis and treatment of the lower urinary tract, lower intestine, pelvic floor, and pathologies related thereto. Pathologies arising in the field of urology, and prostate problems such as prostate enlargement (benign prostatic hyperplasia) and prostate cancer may all benefit from early diagnosis and treatment.

### LUTS

There are a number of disorders involving the lower urinary tract, referred to as Lower Urinary Tract Symptoms involving the bladder, urinary sphincter, urethra, and in men, the prostate.

### Pelvic Floor Dysfunction

The pelvic floor is a group of muscles found in the floor or base of the pelvis, at the bottom of the torso. Pelvic floor dysfunction (PFD) is a common condition where a person is unable to correctly relax and coordinate the muscles in the pelvic floor in order to urinate or to have a bowel movement.

In some cases, symptoms such as problems occurring with bladder or bowel control may be an indication of PFD. In other cases, a healthcare provider may be able to diagnose PFD with a physical exam using their hands to check for spasms, knots or weakness in these muscles. An intrarectal or vaginal exam may be used to diagnose PFD as well.

Some other tests that are generally used for PFD diagnosis are as follows:

Surface electrodes can be used to test pelvic muscle control; the electrodes are placed on the perineum or on the sacrum.

Anorectal manometry can test pressure, muscle strength and coordination.

A defecating proctogram is a test where the patient is given an enema of a thick liquid that can be seen on X-ray. The movement of the intestinal and pelvic muscles are followed with X-ray video during defecation.

A uroflow test tests how well a subject can empty their bladder. If the flow of urine is weak or one has to stop and start during urination, it can be an indication of pelvic floor dysfunction.

### High Intensity Focused Ultrasound

High intensity focused ultrasound (HIFU) is a technique that can be used for a number of applications including increasing flow of blood or lymph, and ablating tissue. This is an emerging technology for minimally invasive or noninvasive surgery, where ultrasound waves are transmitted through the skin into the body and focused into a small region of the body. The intensity of the ultrasound at the focal point generates local heat in the tissue due to absorption processes, and thus causes a local rise in temperature. The technique uses steady or pulsed waves to cause mechanical or thermal ablation of tissue.

HIFU may be used, for example, to treat essential tremor, neuropathic pain, tremor due to Parkinson's disease, uterine adenomyosis and fibroids, palliative treatment for bone metastasis and pancreatic cancer, prostate enlargement, and for cosmetic uses.

Other mechanisms for therapeutic HIFU exist as well, including, for example, mild elevation of temperature, cavitation, hi stotripsy, neural stimulation, sonoporation (changing the porosity of tissue, or the permeability of biological membranes by ultrasound), and drug delivery.

US 2005-0080341 to He describes a seat frame for use with an extracorporeal HIFU therapeutic apparatus, in which the HIFU therapeutic apparatus includes a HIFU source for providing HIFU, a carrying device for the patient and a displacement system for causing spatial movement of the carrying device for the patient with respect to the HIFU source. A containing means is described for receiving the transmission medium in front of an emitting surface of the HIFU source, the containing means for the transmission medium being an open-type water tank. The seat frame includes a cushion which may be submerged in the open-type water tank. In the cushion is an arrangement through which the power ultrasonic waves may pass. The seat frame further includes a cushion supporting arrangement for supporting the cushion on the carrying device for the patient.

WO 98/047570 to Talish describes a system for therapeutically treating injuries using ultrasound. The system is described as including an ergonomically constructed ultrasonic transducer treatment head module and a main operating unit. The transducer treatment head module is positioned adjacent the area of the injury and excited for a predetermined period of time. The system includes a bathtub insert that envelops a portion of the patient's body, and means on the insert for positioning and holding the treatment head module adjacent positions on the patient's body.

WO 2018/015944 to Ben-Ezra, which is incorporated herein by reference, describes apparatus for assessing a characteristic of a first acoustic field at a first frequency in a region of a medium, the first acoustic field generating oscillatory motion of scatterers disposed within the medium, at the first frequency. An acoustic transducer (a) generates a second acoustic field at a second frequency in the region, the second frequency being higher than the first frequency, and (b) receives echo data of the second acoustic field scattering off the oscillating scatterers in the medium, the echo data containing Doppler-shifted frequencies related to the oscillations of the scatterers, resulting in a time-dependent Doppler shift that oscillates at a frequency that is related to the first frequency. Control circuitry (a) extracts the oscillating time-dependent Doppler shift from the received echo data, and (b) converts the extracted Doppler shift into particle-velocity of the first acoustic field.

WO 2019/145945 to Ben-Ezra, which is incorporated herein by reference, describes a first transducer transmitting a first acoustic field at a first frequency into a region of a medium, generating oscillatory motion of scatterers disposed in the region. A second transducer transmits acoustic pulses into the region, and receives respective echoes of each pulse scattering off an oscillating scatterer in the region. The pulses are synchronized with the first acoustic field such that a first pulse scatters off the oscillating scatterer when the scatterer is at a first displacement extremum, and a second pulse scatters off the oscillating scatterer when the scatterer is at a second displacement extremum that is opposite the first displacement extremum. A computer processor extracts a time shift between the received echoes, calculates a displacement amplitude of the scatterer, and outputs an indication of the displacement amplitude of the scatterer.

### SUMMARY OF THE INVENTION

Methods are described and apparatus provided for a system that performs both diagnostic ultrasound and therapeutic ultrasound, in accordance with an application of the present invention. An ultrasound imaging probe generates a sonogram of internal anatomy of the subject by generating an imaging acoustic field, and a high intensity focused ultrasound (HIFU) transducer generates a therapeutic acoustic field by emitting HIFU energy into the subject. Both the ultrasound imaging probe and the HIFU transducer (a) are fixed to a common treatment head, (b) are operated so as to use the perineum of a subject, e.g., a patient, as the acoustic window through which their respective ultrasound energies enter the body of the subject, and (c) are operated to, respectively, generate the sonogram of the internal anatomy and the therapeutic acoustic field while the subject is in a sitting position and is awake, i.e., is not under general anesthesia and is typically not sedated.

Typically, the apparatus includes a chair having a chair-frame and a seat. The chair-frame, the seat, and the treatment head are arranged such that when the subject is sitting in the chair the perineum of the subject is acoustically coupled to the treatment head. Control circuitry (a) controls movement of the treatment head relative to the seat, (b) operates the ultrasound imaging probe to generate at least one sonogram of internal anatomy of the subject through the perineum of the subject, and (c) operates the HIFU transducer to emit HIFU energy into the body of the subject through the perineum of the subject.

For some applications, the subject is sitting in the chair during a diagnostic exam and/or a therapeutic treatment session and a practitioner uses the control circuitry to operate the treatment head, i.e., movement of the treatment head and operation of the ultrasound imaging probe and/or the HIFU transducer, from a location that is remote from the chair on which the subject is sitting. For example, the practitioner may be operating the treatment head from a desktop computer that is disposed in a separate room than where the subject is sitting on the chair, or in the same room yet at a distance from the subject, allowing the subject some degree of privacy during the exam and/or treatment session.

Conventionally, trans-perineal ultrasound is performed by a practitioner holding an ultrasound probe against the perineum of a patient while the patient is lying on their back, and often under sedation or anesthesia due to expected discomfort during the exam and/or treatment session, or in order to prevent the patient from moving during the exam and/or treatment. Prostate exams are conventionally performed trans-rectally, often under sedation or anesthesia. Applications of the present invention allow for the patient to undergo trans-perineal pelvic floor diagnostic and/or therapeutic ultrasound while sitting in a comfortable position and being awake, thereby providing certain advantages. For example, a non-limiting list is as follows:

being able to sit provides the patient with comfort,

being in a sitting position, as opposed to supine, provides an improved geometry of the anatomy of the pelvic floor and nearby organs for ultrasound imaging,

for the purposes of a urological exam and urodynamics, the patient is able to urinate during the exam due to being awake and sitting, allowing the practitioner to visualize the flow of the urine from the bladder along the urethra,

pelvic floor muscle contractions can be observed, as well as the functioning of the patient's sphincters,

other biological flows such as the flow of bowel during defecation, or the flow of semen during ejaculation can be observed, and/or

ease and comfort of the exam and/or treatment session may encourage patients to go for routine examinations that they may otherwise have pushed off due to the conventional discomfort of such exams, e.g., routine prostate exams.

For some applications, a flexible membrane is sealably coupled to a perimeter of a housing of the treatment head, the flexible membrane and the housing forming an internal cavity that may be filled with a liquid. When the internal cavity is filled with liquid the ultrasound imaging probe and the HIFU transducer are in direct contact with the liquid. The flexible membrane is sized and shaped such that it inflates outwards from the perimeter of the housing due to pressure from the liquid within the internal cavity. Thus, when the subject is sitting in the chair the perineum of the subject is acoustically coupled to the treatment head via the flexible membrane, the flexible membrane being pressed against the perineum of the subject due to the pressure. Acoustic coupling of the perineum to the treatment head via the inflated membrane (a) helps to tighten the skin of the perineum due to the pressure, thereby providing improved acoustic coupling, and (b) provides a liquid-filled space between the treatment head and the skin of the perineum such that the treatment head may be moved with 3-6 degrees of freedom relative to the seat of the chair while maintaining the acoustic coupling between the perineum and the treatment head. The inflated flexible membrane also provides comfort to the patient—the patient experiences the sensation of a small pillow being pressed against the perineum instead of a conventional ultrasound probe being pushed against the skin, or a transrectal examination.

There is therefore provided, in accordance with some applications of the present invention apparatus for use with a subject, the apparatus including:

a treatment head including:
an ultrasound imaging probe configured to generate a sonogram of internal anatomy of the subject by generating an imaging acoustic field; and
a high intensity focused ultrasound (HIFU) transducer configured to generate a therapeutic acoustic field by emitting HIFU energy into the subject;

a chair including a chair-frame and a seat, wherein the chair-frame, the seat, and the treatment head are arranged such that when the subject is sitting in the chair a perineum of the subject is acoustically coupled to the treatment head; and

control circuitry configured to (a) control movement of the treatment head relative to the seat, (b) operate the ultrasound imaging probe to generate at least one sonogram of internal anatomy of the subject through the perineum of the subject, and (c) operate the HIFU transducer to emit HIFU energy into the body of the subject through the perineum of the subject.

For some applications, the treatment head is moveably coupled to the chair-frame.

For some applications, the HIFU transducer and the ultrasound imaging probe are fixed to the treatment head such that movement of the treatment head relative to the seat moves the HIFU transducer and the ultrasound imaging probe relative to the seat.

For some applications, the control circuitry is configured to control translation of the treatment head along a longitudinal axis of the treatment head, and along an axis that is perpendicular to the longitudinal axis.

For some applications, the control circuitry is configured to control rotation of the treatment head about an axis that is perpendicular to a longitudinal axis of the treatment head.

For some applications, the control circuitry is configured to control rotation of the treatment head about a longitudinal axis of the treatment head.

For some applications, the ultrasound imaging probe is configured to rotate relative to the treatment head about a longitudinal axis of the treatment head, and wherein the control circuitry is further configured to control the rotation of the ultrasound imaging probe relative to the treatment head.

For some applications, the HIFU transducer and the ultrasound imaging probe are coaxial, and the ultrasound imaging probe is configured to rotate within a central bore of the HIFU transducer.

For some applications, the treatment head includes:

a housing, in which the ultrasound imaging probe and the HIFU transducer are disposed; and

a flexible membrane sealably coupled to a perimeter of the housing, the flexible membrane and the housing forming an internal cavity that is configured to be filled with a liquid such that, when the internal cavity of the housing is filled with the liquid the ultrasound imaging probe and the HIFU transducer are in direct contact with the liquid:
the flexible membrane being configured to inflate outwards from the perimeter of the housing due to pressure from the liquid within the internal cavity, and
when the subject is sitting in the chair the perineum of the subject being acoustically coupled to the treatment head via the flexible membrane, the flexible membrane being pressed against the perineum of the subject due to the pressure.

For some applications, the liquid is degassed water.

For some applications, at least a portion of the flexible membrane is water permeable and is configured such that when the flexible membrane is pressed against the perineum of the subject, liquid from within the internal cavity of the housing seeps through the at least a portion of the flexible membrane such that the perineum is acoustically coupled to the flexible membrane via the liquid that seeped through the at least a portion of the flexible membrane.

For some applications, the flexible membrane is arranged such that a major axis of a projection of the uninflated flexible membrane taken along a longitudinal axis of the treatment head of is 6-12 cm.

For some applications, the flexible membrane is arranged such that the major axis of the projection of the uninflated flexible membrane taken along the longitudinal axis of the treatment head is 2-8 cm longer than a major axis of the perimeter of the housing to which the flexible membrane is sealably coupled.

For some applications, the flexible membrane is arranged such that the major axis of the projection of the uninflated flexible membrane taken along the longitudinal axis of the treatment head is 20-100% larger than a major axis of the perimeter of the housing to which the flexible membrane is sealably coupled.

For some applications, the apparatus further includes a pressure regulator coupled to the housing and configured to regulate the pressure within the internal cavity of the housing.

For some applications:

the housing includes a fluid port in fluid communication with the internal cavity of the housing, and

the pressure regulator is configured to regulate the pressure within the internal cavity of the housing by regulating a volume of the liquid within the internal cavity of the housing using the fluid port.

For some applications:

the fluid port is a first fluid port and the housing further includes a second fluid port, (a) the first fluid port being a fluid inlet port through which the liquid is received into the internal cavity of the housing, (b) the second fluid port being a fluid outlet port through which the liquid is drained from the internal cavity of the housing, and

the pressure regulator is configured to regulate the pressure within the internal cavity of the housing by regulating a volume of the liquid within the internal cavity of the housing using the fluid inlet port and the fluid outlet port.

For some applications, the flexible membrane is coupled to the housing such that (a) when the flexible membrane is inflated by the internal cavity of the housing being filled with a volume of liquid such that the pressure within the internal cavity is 1.2 atm, and (b) the subject is not sitting in the chair, an uncompressed height of the inflated flexible membrane along a longitudinal axis of the treatment head is 2-12 cm.

For some applications, the pressure regulator is configured such that, when the subject is sitting in the chair, the pressure regulator maintains the flexible membrane pressed against the perineum of the subject by maintaining the pressure within the internal cavity at an operational pressure.

For some applications, the operational pressure is 1.2-2 atm.

For some applications, the pressure regulator is configured such that, when the subject is sitting in the chair, during motion of the treatment head relative to the seat, the pressure regulator maintains the flexible membrane pressed against the perineum of the subject by maintaining the pressure within the internal cavity at the operational pressure.

For some applications, the flexible membrane and the housing of the treatment head are arranged such that when (a) the subject is sitting in the chair and (b) the pressure regulator is maintaining the pressure at the operational pressure during the motion of the treatment head relative to the seat, the treatment head can translate along the longitudinal axis of the treatment head at least 1 cm away from the perineum of the subject without the flexible membrane losing contact with the perineum of the subject.

For some applications, the flexible membrane and the housing of the treatment head are arranged such that when (a) the subject is sitting in the chair and (b) the pressure regulator is maintaining the pressure at the operational pressure during the motion of the treatment head relative to the seat, the treatment head can translate at least 1 cm along an axis that is perpendicular to the longitudinal axis of the treatment head without a contact portion of the flexible membrane that is in contact with the perineum of the subject sliding with respect to the perineum of the subject.

For some applications, the flexible membrane and the housing of the treatment head are arranged such that when (a) the subject is sitting in the chair and (b) the pressure regulator is maintaining the pressure at the operational pressure during the motion of the treatment head relative to the seat, the treatment head can rotate by at least 5 degrees about an axis that is perpendicular to the longitudinal axis of the treatment head without a contact portion of the flexible membrane that is in contact with the perineum of the subject sliding with respect to the perineum of the subject.

For some applications, the apparatus further includes a robotic arm, the treatment head being coupled to a distal end of the robotic arm, wherein the control circuitry is configured to control movement of the treatment head relative to the seat by controlling movement of the robotic arm.

For some applications, a proximal end of the robotic arm is coupled to the chair-frame.

For some applications, the robotic arm is configured to move the treatment head in a plurality of degrees of freedom.

For some applications, the HIFU transducer and the ultrasound imaging probe are fixed to the treatment head such that movement of the treatment head relative to the seat moves the HIFU transducer and the ultrasound imaging probe relative to the seat.

For some applications, the robotic arm is configured to translate the treatment head along a longitudinal axis of the treatment head, and along an axis that is perpendicular to the longitudinal axis.

For some applications, the robotic arm is configured to rotate the treatment head about an axis that is perpendicular to a longitudinal axis of the treatment head.

For some applications, the robotic arm is configured to rotate the treatment head about a longitudinal axis of the treatment head.

For some applications, the ultrasound imaging probe is configured to rotate relative to the treatment head about a longitudinal axis of the treatment head, and wherein the robotic arm is further configured to control the rotation of the ultrasound imaging probe relative to the treatment head.

For some applications, the HIFU transducer and the ultrasound imaging probe are coaxial, and the ultrasound imaging probe is configured to rotate within a central bore of the HIFU transducer.

For some applications, the control circuitry is configured to register the imaging acoustic field and the therapeutic acoustic field, such that the imaging acoustic field and the therapeutic acoustic field share a common coordinate system.

For some applications, the apparatus is for use with a display and, due to the registration of the imaging acoustic field and the therapeutic acoustic field, the control circuitry is configured to show on the display (a) the sonogram of the internal anatomy of the subject and (b) a focal region of the HIFU energy with respect to the internal anatomy of the subject overlaid on the sonogram of the internal anatomy of the subject on the display.

For some applications, the display is a first display configured to be used by a practitioner, the apparatus is further for use with a second display disposed such that the second display is visible to the subject when the subject is sitting in the chair, and the control circuitry is configured to display on the second display spatial information based on (a) the sonogram of the internal anatomy of the subject and (b) a focal region of the HIFU energy with respect to the internal anatomy of the subject.

For some applications, the control circuitry is configured to display the spatial information by displaying a spatial relationship between internal anatomy of the subject and the focal region of the HIFU energy with respect to the internal anatomy of the subject.

For some applications, the control circuitry is configured to display (a) the sonogram of the internal anatomy of the subject and (b) a focal region of the HIFU energy with respect to the internal anatomy of the subject overlaid on the sonogram of the internal anatomy of the subject.

For some applications, the second display is coupled to the chair-frame.

For some applications, the control circuitry is configured to display an indication to the subject relating to a progression of a procedure that the subject is undergoing, the procedure being performed via the treatment head.

For some applications, the control circuitry is configured to display the indication by displaying an alert to the subject on the second display if the focal region of the HIFU energy has moved with respect to internal anatomy of the subject due to movement of the subject with respect to the seat.

For some applications, the control circuitry includes user controls configured to be used by the subject when the subject is sitting in the chair in order to provide feedback to the practitioner relating to a sensation that the subject is experiencing.

For some applications, the user controls include a stop-actuator which the subject can actuate while sitting in the chair, the stop-actuator being configured to terminate the emission of HIFU energy into the subject.

For some applications, the treatment head includes:

a housing, in which the ultrasound imaging probe and the HIFU transducer are disposed; and

a flexible membrane sealably coupled to a perimeter of the housing, the flexible membrane and the housing forming an internal cavity that is filled with a volume of liquid such that the ultrasound imaging probe and the HIFU transducer are in direct contact with the liquid, wherein:
the flexible membrane is inflated outwards from the perimeter of the housing due to pressure from the liquid within the internal cavity, and
when the subject is sitting in the chair the perineum of the subject is acoustically coupled to the treatment head via the flexible membrane, the flexible membrane being pressed against the perineum of the subject due to the pressure.

For some applications, at least a portion of the flexible membrane is water permeable and is configured such that when the flexible membrane is pressed against the perineum of the subject, liquid from within the internal cavity of the housing seeps through the at least a portion of the flexible membrane such that the perineum is acoustically coupled to the flexible membrane via the liquid that seeped through the at least a portion of the flexible membrane.

For some applications, the flexible membrane is coupled to the housing such that the pressure within the internal cavity is 1.2-2 atm, and is configured such that when the subject is not sitting in the chair, an uncompressed height of the inflated flexible membrane along a longitudinal axis of the treatment head is 2-4 cm.

For some applications, the flexible membrane is arranged such that a major axis of a projection of the inflated flexible membrane taken along a longitudinal axis of the treatment head when the subject is not sitting in the chair of is 6-12 cm.

For some applications, the flexible membrane is arranged such that the major axis of the projection of the inflated flexible membrane taken along the longitudinal axis of the treatment head when the subject is not sitting in the chair is 2-8 cm longer than a major axis of the perimeter of the housing to which the flexible membrane is sealably coupled.

For some applications, the flexible membrane is arranged such that the major axis of the projection of the inflated flexible membrane taken along the longitudinal axis of the treatment head when the subject is not sitting in the chair is 20-100% larger than a major axis of the perimeter of the housing to which the flexible membrane is sealably coupled.

For some applications, the control circuitry is configured such that, when the subject is sitting in the chair, during motion of the treatment head relative to the seat, the control circuitry maintains the flexible membrane pressed against the perineum of the subject.

For some applications, the control circuitry is configured such that, when the subject is sitting in the chair, during motion of the treatment head relative to the seat, the control circuitry regulates the pressure within the internal cavity by regulating a force with which the flexible membrane is pressed against the perineum.

For some applications, the flexible membrane and the housing of the treatment head are arranged such that when (a) the subject is sitting in the chair and (b) the control circuitry is maintaining the flexible membrane pressed against the perineum of the subject during motion of the treatment head relative to the seat, the treatment head can translate at least 1 cm along an axis that is perpendicular to the longitudinal axis of the treatment head without a contact portion of the flexible membrane that is in contact with the perineum of the subject sliding with respect to the perineum of the subject.

For some applications, the flexible membrane and the housing of the treatment head are arranged such that when (a) the subject is sitting in the chair and (b) the control circuitry is maintaining the flexible membrane pressed against the perineum of the subject during motion of the treatment head relative to the seat, the treatment head can rotate by at least 5 degrees about an axis that is perpendicular to the longitudinal axis of the treatment head without a contact portion of the flexible membrane that is in contact with the perineum of the subject sliding with respect to the perineum of the subject.

For some applications, the flexible membrane is an elastic membrane, and wherein the elastic membrane is (a) coupled to the housing such that the pressure within the internal cavity is 1.2-2 atm, and (b) configured such that when the subject is not sitting in the chair, an uncompressed height of the inflated elastic membrane along a longitudinal axis of the treatment head is 2-12 cm.

For some applications, the elastic membrane is arranged such that a major axis of a projection of the inflated elastic membrane taken along a longitudinal axis of the treatment head when the subject is not sitting in the chair of is 6-12 cm.

For some applications, the elastic membrane is arranged such that the major axis of the projection of the inflated elastic membrane taken along the longitudinal axis of the treatment head when the subject is not sitting in the chair is 2-8 cm longer than a major axis of the perimeter of the housing to which the elastic membrane is sealably coupled.

For some applications, the elastic membrane is arranged such that the major axis of the projection of the inflated elastic membrane taken along the longitudinal axis of the treatment head when the subject is not sitting in the chair is 20-100% larger than a major axis of the perimeter of the housing to which the elastic membrane is sealably coupled.

For some applications, the control circuitry is configured such that, when the subject is sitting in the chair, during motion of the treatment head relative to the seat, the control circuitry maintains the elastic membrane pressed against the perineum of the subject.

For some applications, the control circuitry is configured such that, when the subject is sitting in the chair, during motion of the treatment head relative to the seat, the control circuitry regulates the pressure within the internal cavity by regulating a force with which the elastic membrane is pressed against the perineum.

For some applications, the elastic membrane and the housing of the treatment head are arranged such that when (a) the subject is sitting in the chair and (b) the control circuitry is maintaining the elastic membrane pressed against the perineum of the subject during motion of the treatment head relative to the seat, the treatment head can translate along the longitudinal axis of the treatment head at least 1 cm away from the perineum of the subject without the elastic membrane losing contact with the perineum of the subject.

For some applications, the elastic membrane and the housing of the treatment head are arranged such that when (a) the subject is sitting in the chair and (b) the control circuitry is maintaining the elastic membrane pressed against the perineum of the subject during motion of the treatment head relative to the seat, the treatment head can translate at least 1 cm along an axis that is perpendicular to the longitudinal axis of the treatment head without a contact portion of the elastic membrane that is in contact with the perineum of the subject sliding with respect to the perineum of the subject.

For some applications, the elastic membrane and the housing of the treatment head are arranged such that when (a) the subject is sitting in the chair and (b) the control circuitry is maintaining the elastic membrane pressed against the perineum of the subject during motion of the treatment head relative to the seat, the treatment head can rotate by at least 5 degrees about an axis that is perpendicular to the longitudinal axis of the treatment head without a contact portion of the elastic membrane that is in contact with the perineum of the subject sliding with respect to the perineum of the subject.

The present invention will be more fully understood from the following detailed description of applications thereof, taken together with the drawings, in which:

### BRIEF DESCRIPTION OF THE DRAWINGS

FIG. 1 is a schematic illustration of apparatus for pelvic floor diagnosis and treatment, in accordance with some applications of the present invention;

FIGS. 2A-D are schematic illustrations of a treatment head in accordance with some applications of the present invention;

FIGS. 3A-D are schematic illustrations of an ultrasound imaging probe and a HIFU transducer within the treatment head of FIGS. 2A-D, in accordance with some applications of the present invention;

FIGS. 4A-E are schematic illustrations of a treatment head and a flexible membrane sealably coupled to a perimeter of a housing of the treatment head, in accordance with some applications of the present invention;

FIGS. 4F-H are schematic illustrations of a treatment head, a housing, and a flexible membrane in accordance with some applications of the present invention; FIGS. 5A-D are schematic illustrations of the treatment head and the inflated flexible membrane during motion of the treatment head, in accordance with some applications of the present invention;

FIGS. 5A-D are schematic illustrations of the treatment head and inflated flexible membrane during motion of the treatment head, in accordance with some applications of the present invention;

FIG. 6 is a schematic illustration of apparatus for pelvic floor diagnosis and treatment, in accordance with some applications of the present invention;

FIGS. 7A-F are schematic illustrations of a display that is visible to a subject undergoing an exam and/or treatment session with the apparatus of FIGS. 1-6, in accordance with some applications of the present invention; and

FIGS. 8A-B are schematic illustrations of the treatment head, in accordance with some applications of the present invention.

### DETAILED DESCRIPTION

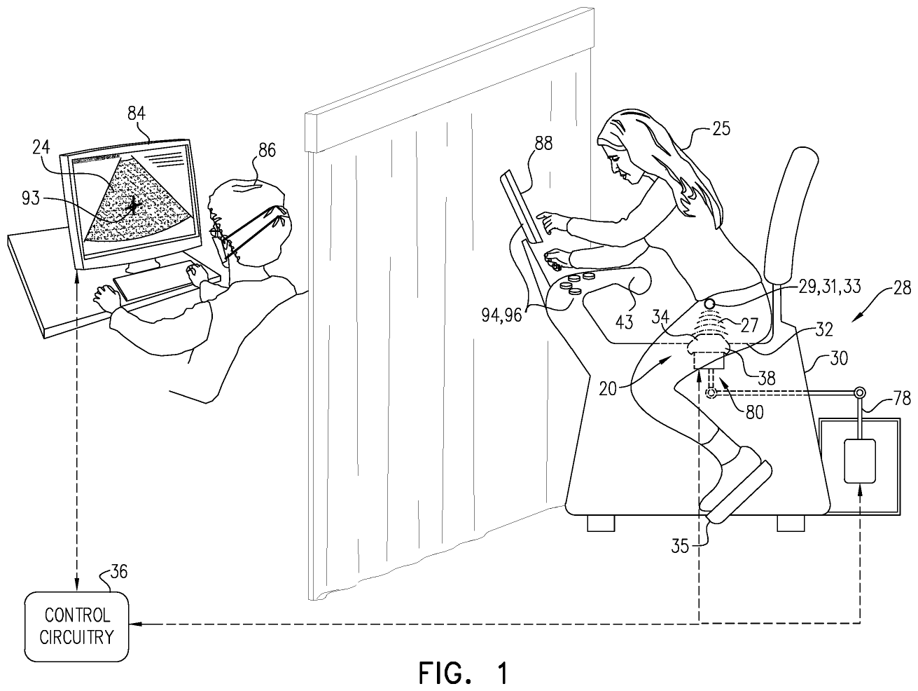

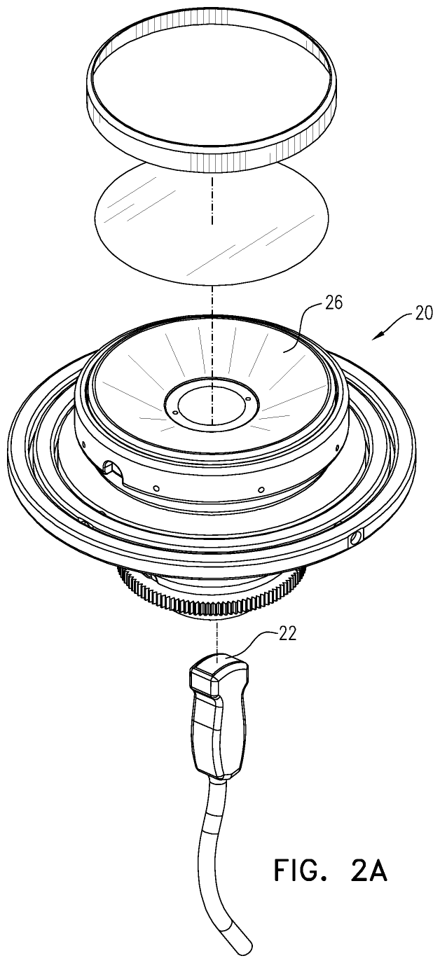

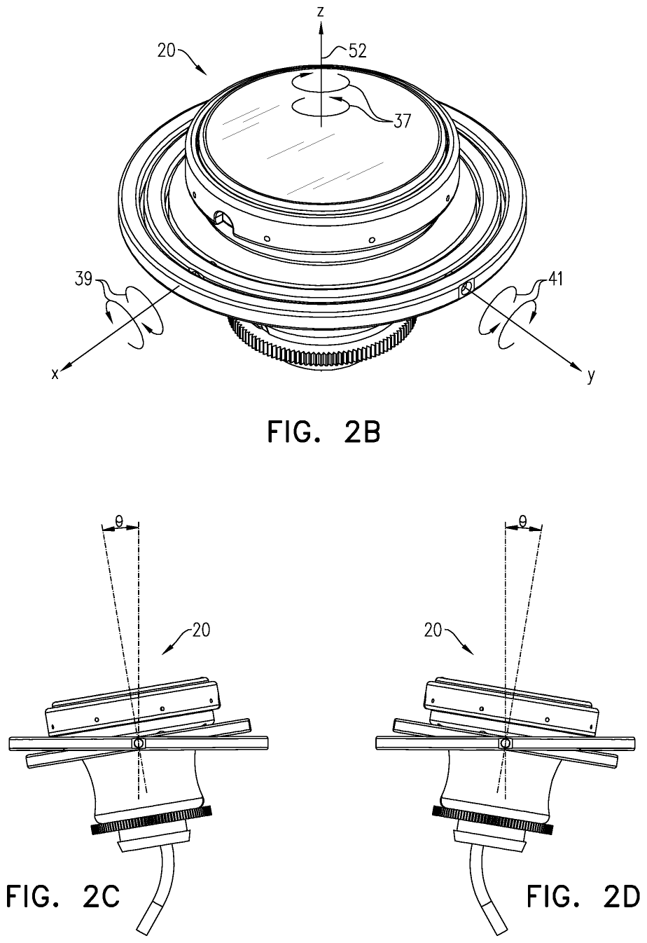

Reference is now made to FIG. 1, which is a schematic illustration of apparatus for pelvic floor diagnosis and treatment, in accordance with some applications of the present invention. A treatment head 20 includes an ultrasound imaging probe 22 (shown, for example, in FIG. 2A) which generates a sonogram 24 of internal anatomy of a subject 25 by generating an imaging acoustic field 27, and a high intensity focused ultrasound (HIFU) transducer 26 (shown, for example, in FIG. 2A) which generates a therapeutic acoustic field 29 by emitting HIFU energy 31 into subject 25. Sonogram 24 and an image 93 of therapeutic acoustic field 29 are shown on a display 84 of a practitioner 86, further described hereinbelow. A chair 28 is provided for subject 25 to sit on, chair 28 including a chair-frame 30 and a seat 32. Chair-frame 30, seat 32, and treatment head 20 are arranged such that when subject 25 is sitting in chair 28 a perineum 34 of subject 25 is acoustically coupled to treatment head 20. Control circuitry 36 (a) controls movement of treatment head 20 relative to seat 32, (b) operates ultrasound imaging probe 22 to generate at least one sonogram 24 of internal anatomy of subject 25 through perineum 34 of subject 25, and (c) operates HIFU transducer 26 to emit HIFU energy into the body of subject 25 through perineum 34 of subject 25. For some applications, treatment head 20 is moveably coupled to chair-frame 30. Alternatively, treatment head 20 may be separate from chair-frame 30. For some applications, chair-frame may include foot rests 35 and/or arm rests 43 in order to increase the comfort of chair 28 for subject 25. Having somewhere for subject 25 to brace their arms and legs may also help them to remain still during the exam and/or treatment session, and in particular during the emission of HIFU energy 31.

Reference is now made to FIGS. 2A-D, which are schematic illustrations of treatment head 20 in accordance with some applications of the present invention. For some applications, HIFU transducer 26 and ultrasound imaging probe 22 are fixed to treatment head 20 such that movement of treatment head 20 relative to seat 32 moves HIFU transducer 26 and ultrasound imaging probe 22 relative to seat 32. FIG. 2A shows an exploded view of treatment head 20 with HIFU transducer 26 and ultrasound imaging probe 22. FIG. 2B shows treatment head 20 along with three axes of treatment head 20: a longitudinal axis (z-axis) 52, an x-axis, and a y-axis, the x and y axes both being perpendicular to longitudinal axis 52. Typically, control circuitry 36 controls movement of treatment head 20 by controlling:

translation of treatment head 20 along longitudinal axis 52 of the treatment head, e.g., the z-axis as shown in FIG. 2B, and along an axis that is perpendicular to the longitudinal axis, e.g., the x-axis and/or the y-axis as shown in FIG. 2B;

rotation of treatment head 20 about an axis that is perpendicular to longitudinal axis 52 of the treatment head, i.e., tilting of treatment head 20 as shown in FIGS. 2C and 2D, as illustrated by rotation arrows 39 and 41 in FIG. 2B;

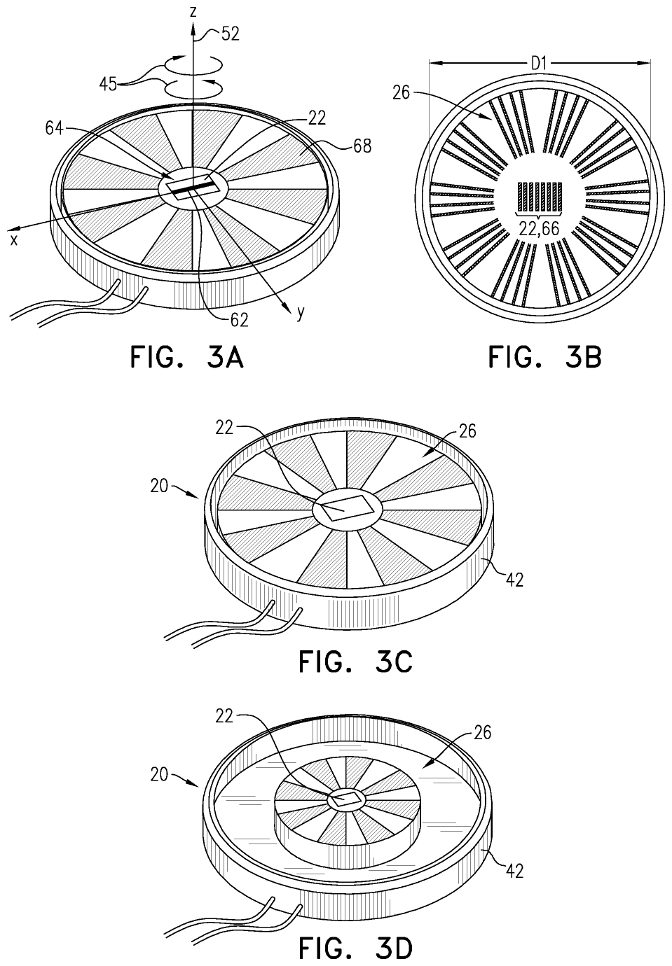

rotation of treatment head 20 about longitudinal axis 52 of treatment head 20, as illustrated by rotation arrows 37 in FIG. 2B.
Reference is now made to FIGS. 3A-D, which are schematic illustrations of ultrasound imaging probe 22 and HIFU transducer 26 within treatment head 20, in accordance with some applications of the present invention. Typically, viewing the prostate from the perineum requires a field of view (FOV) of approximately 100 mm depth, with a sector angle of about +/−20 degrees. Ultrasound images of the prostate may include the lower part of the bladder, the urethra, the entire prostate, seminal vesicles, penile bulb, and more. A typical frequency for ultrasound imaging probe 22 may be 3-7.5 MHz. For some applications, ultrasound imaging probe 22 has a 1D linear array 61 of piezoelectric elements, e.g., 64 or 128 elements in a row. In principle, a 1D array of elements (e.g., a linear or a curvilinear array of elements) provides a 2D image. For some applications, in order to achieve 3D imaging of the anatomical region of interest, ultrasound imaging probe 22 control circuitry 36 rotates ultrasound imaging probe 22 relative to treatment head 20 about longitudinal axis 52 of treatment head 20, as illustrated in FIG. 3A. The rotation of ultrasound imaging probe 22 relative to treatment head is illustrated by rotation arrows 45. This enables a series of 2D ultrasound images, i.e., slices, to be captured, which can then be reconstructed into a 3D visualization of the region (e.g., the prostate and its surroundings), thus implementing ultrasonic tomography. For some applications HIFU transducer 26 and ultrasound imaging probe 22 are coaxial, and ultrasound imaging probe 22 is configured to rotate within a central bore 64 of the HIFU transducer. Alternatively or additionally, ultrasound imaging probe 22 may have a 2D array 66 of ultrasound elements, e.g., 8 linear arrays of 64 elements each (arranged side by side), as illustrated in FIG. 3B. With 2D array 66 of piezoelectric elements 3D, ultrasound imaging of the anatomical region of interest is possible using phased array electronic control.

For some applications, real-time tracking using ultrasound imaging probe 22 may be employed by continuous imaging of the anatomical region of interest, for example at a frame rate of 15 images per second. This enables tracking the position of the anatomy of interest, e.g., the prostate, in real time for 4D ultrasonic tomography. Real-time tracking allows the system to compensate for a patient's movements as well as any natural physiological motions such as, for example, motion due to breathing, bowel activity, intestinal activity, and blood flow. Real-time tracking is also useful for monitoring the patient's movements in order to analyze them and respond accordingly, e.g., to stop the emission of HIFU energy if the patient is moving too much.

HIFU transducer 26 may be based on piezoelectric elements made of PZT (for example, PZT-4, PZT-8, and the like), and may be constructed in one of a plurality of common designs for HIFU transducers, e.g., using a single element (in the form of a focusing bowl, spherical shell, or other form), an annular array (for example in the form of concentric rings), a phased array design (using large number of small pixel-like elements), or any combination of the above designs. The frequency of the therapeutic ultrasound is selected according to the intended depth of penetration into the body of subject 25, the attenuation, and the intended effect at the focal point. For example, a characteristic selection for hyperthermal ablation in soft tissue may be in the range of 0.5-4.0 MHz, depending on the distance to the target and other factors.

For some applications, treatment head 20 may be trimodal, providing the following three functions: (i) HIFU energy, (ii) ultrasound imaging, and (iii) visualization of the HIFU acoustic field with ultrasound imaging. The visualization of the HIFU acoustic field with ultrasound imaging may be done using techniques described in US 2019/0232090 to Ben-Ezra and US 2021/0045714 to Ben-Ezra, both of which are incorporated herein by reference.

In the specific example shown in FIGS. 3A-D, HIFU transducer 26 has ten identical sectors 68 arranged in a circle around the center within a housing 42 of treatment head 20. The outer diameter D1 of HIFU transducer 26 is 85 mm in this example, as shown in FIG. 3B. For some applications, outer diameter D1 of HIFU transducer 26 is at least 40 mm and/or less than 120 mm. Each sector 68 is a multi-element phased array HIFU transducer with N elements (for example, a square matrix with 9×9 elements, each element being 1.8×1.8 mm, the kerf between elements being 0.2 mm, N=81, and the size of a single sector 68 is of the order of 20 mm). The ten sectors 68 together form HIFU transducer 26. For some applications, HIFU transducer 26 focuses to a distance of 75 mm (with a shared focal point for each of the ten sectors 68). A typical frequency is 1.2 MHz with a bandwidth (−3 dB) of +/−0.2 MHz. In this design electronic beam forming is used for steering only. All the elements are controlled separately and independently by the system, i.e., each element is operated by a dedicated channel. For example, the driving signal for element k may be a continuous sine wave (CW) with amplitude Ak, frequency Fk (usually, the frequency will be identical for all elements), and phase Pk. In this example ten sectors 68 of 81 elements results in an 810-channel HIFU system.

One possible design of HIFU transducer 26 is flat, such as is shown in FIGS. 3A-D, where all the elements are on the same plane, radiating upwards. The focusing and steering capabilities are achieved by electronic beam forming. In accordance with another application of the present invention, the ten sectors 68 are flat, but each of them is tilted towards a common focal point (all the central-orthogonal lines intersect at the focal point; the central-orthogonal line is the normal to the sector plane that crosses the center of the sector). In another design, the sectors are spherical shells, and therefore all the elements are arranged on a common spherical shell, with the center of the sphere forming the natural (geometric) focal point.

In FIGS. 3C-D, HIFU transducer 26 and ultrasound imaging probe 22 are shown disposed within housing 42, in accordance with some applications of the present invention. For some applications, housing 42 has a diameter that is similar to an outer diameter of HIFU transducer 26, such as is shown in FIG. 3C. For some applications, housing 42 has a larger diameter than an outer diameter of HIFU transducer 26, such as is shown in FIG. 3D. Housing 42 is further described hereinbelow.

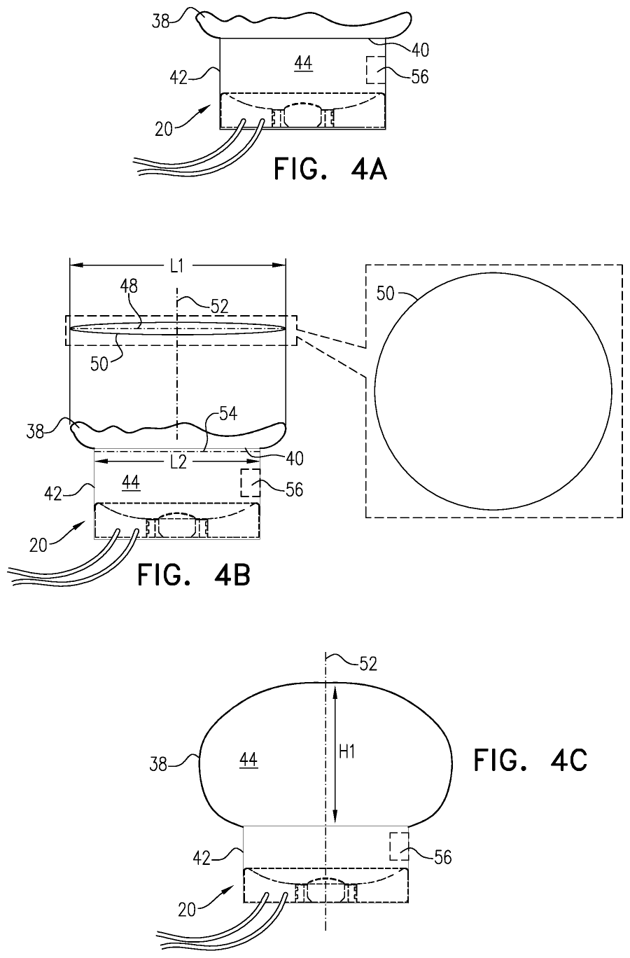

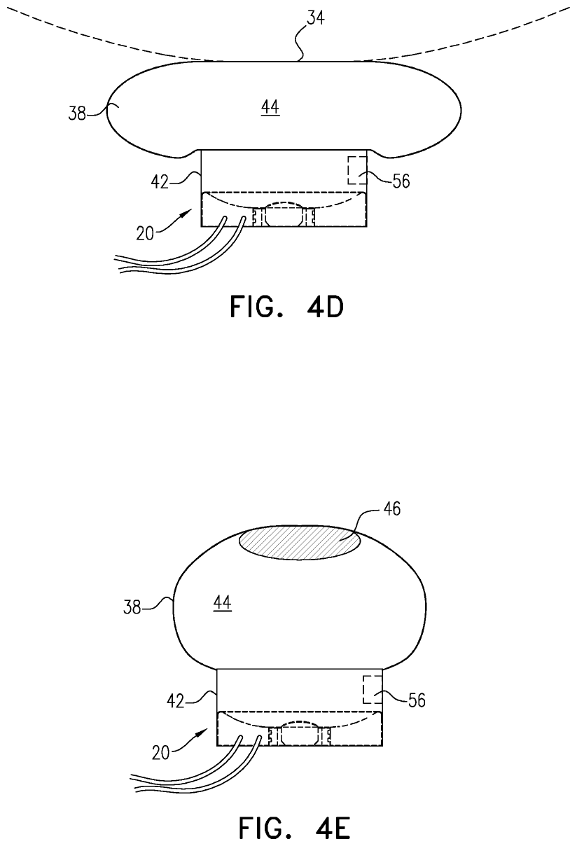

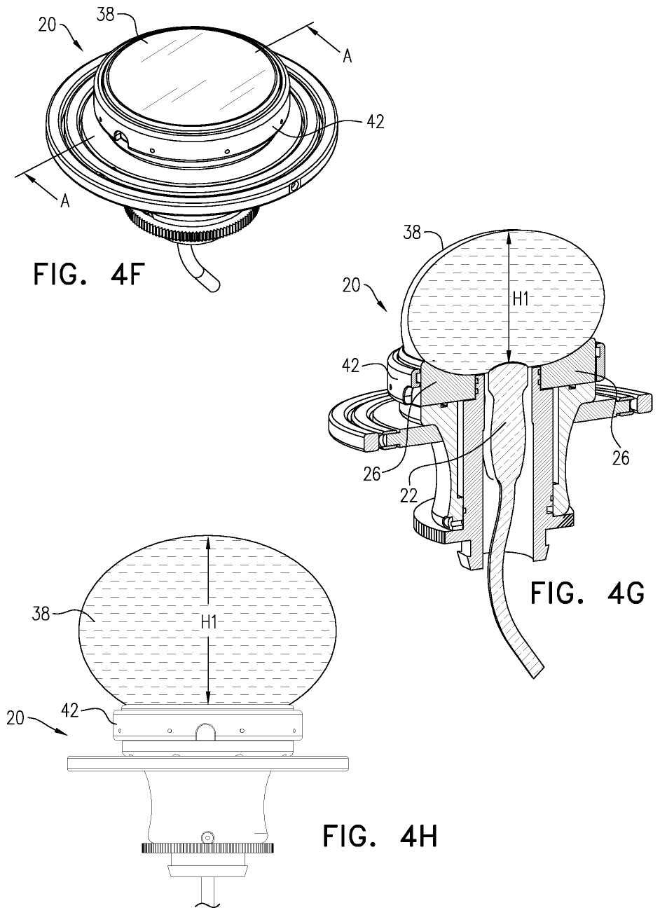

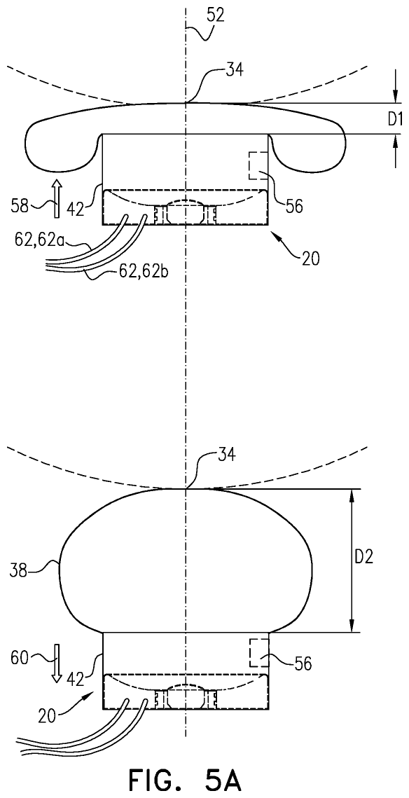

Reference is now made to FIGS. 4A-E, which are schematic illustrations of treatment head 20 and a flexible membrane 38 sealably coupled to a perimeter 40 of a housing 42 of treatment head 20, in accordance with some applications of the present invention. Ultrasound imaging probe 22 and HIFU transducer 26 are disposed within housing 42. Flexible membrane 38 and housing 42 form an internal cavity 44 that is configured to be filled with a liquid, e.g., degassed water, oil, or other known ultrasound coupling liquid. When internal cavity 44 is filled with liquid, ultrasound imaging probe 22 and HIFU transducer 26 are in direct contact with the liquid. Flexible membrane 38 is configured to inflate outwards, such as is shown in FIG. 4C, from perimeter 40 of housing 42 due to pressure from the liquid within internal cavity 44. When subject 25 is sitting in chair 28, perineum 34 of subject 25 is acoustically coupled to treatment head 20 via flexible membrane 38, flexible membrane 38 being pressed against perineum 34 of subject 25 due to the pressure, such as is shown in FIG. 4D. For some applications, flexible membrane 38 is made out of a thermoplastic polyurethane (TPU) material, with a thickness of at least 40 micrometers and/or less than 100 micrometers. Typically, the material of flexible membrane 38 is biocompatible and transparent to ultrasound so as to reduce ultrasonic reflections from the interface between flexible membrane 38 and perineum 34.

For some applications, flexible membrane 38 is acoustically coupled to perineum 34 via an ultrasound coupling gel that may be placed on the upper surface of flexible membrane 38 so as to be in direct contact with the skin of perineum 34. Alternatively, at least a portion 46 of flexible membrane 38 is water permeable and is configured such that when flexible membrane 38 is pressed against perineum 34 of subject 25, liquid from within internal cavity 44 of housing 42 seeps through portion 46 of flexible membrane 38 such that perineum 34 is wetted and acoustically coupled to flexible membrane 38 via the liquid that seeped through portion 46 of flexible membrane 38. For some applications, portion 46 of flexible membrane 38 is a hydrogel membrane, or a TPU material that is water permeable. Portion 46 of flexible membrane 38 is typically an upper portion of flexible membrane 38, such as is shown in FIG. 4E, which comes into contact with perineum 34 of subject 25 when subject 25 is sitting in chair 28. For some applications, the entire flexible membrane 38 may be made of a water permeable material.

Typically, flexible membrane 38 is arranged, e.g., sized and shaped, such that a major axis 48 of a projection 50, taken along a longitudinal axis 52 of treatment head 20, of flexible membrane 38 when flexible membrane 38 is uninflated, has a length L1 that is at least 6 cm and/or less than 12 cm. Typically, length L1 of major axis 48 is (a) at least 2 cm and/or less than 8 cm longer, and/or (b) at least 20% and/or less than 100% larger than a major axis 54 of perimeter 40 of housing 42, as illustrated in FIG. 4B. Major axis 54 of perimeter 40 of housing 42 typically has a length L2 that is at least 4 cm and/or less than 12 cm. As will be further described hereinbelow, flexible membrane 38 being larger than perimeter 40 of housing 42 provides enough space when flexible membrane 38 is inflated such that treatment head 20 may be moved with 3-6 degrees of freedom relative to seat 32 of chair 28 (as described hereinabove with reference to FIGS. 2B-D, and further described hereinbelow) while maintaining the acoustic coupling between perineum 34 of subject 25 and treatment head 20. Typically, flexible membrane 38 is coupled to housing 42 such that (a) when flexible membrane 38 is inflated by internal cavity 44 of housing 42 being filled with a volume of liquid such that the pressure within internal cavity 44 is 1.2 atm, and (b) subject 25 is not sitting in chair 28, an uncompressed height H1 of inflated flexible membrane 38 along longitudinal axis 52 of treatment head 20 is at least 2 cm and/or less than 12 cm, as illustrated in FIG. 4C.

For some applications, a pressure regulator 56 is coupled to housing 42, e.g., disposed within housing 42, and configured to regulate the pressure within internal cavity 44 of housing 42. When subject 25 is sitting in chair 28, pressure regulator 56 maintains flexible membrane 38 pressed against perineum 34 of subject 25 by maintaining the pressure within internal cavity 44 at an operational pressure. Typically, the operational pressure is at least 1.2 atm and/or less than 2 atm.

FIGS. 4F-H show schematic illustrations of treatment head 20, housing 42, and flexible membrane 38 in accordance with some applications of the present invention. FIG. 4G shows cross-section A-A, in which ultrasound imaging probe 22 can be seen coaxially disposed within the central bore of HIFU transducer 26. For some applications, flexible membrane 38 when uninflated is pulled taut like a drum over perimeter 40 of housing 42, such as is shown in FIG. 4F. Alternatively, flexible membrane 38 is larger than perimeter 40 of housing 42, such as described hereinabove with reference to FIGS. 4A-D. In FIGS. 4G and 4H, flexible membrane 38 is shown fully inflated with liquid such that the pressure within internal cavity 44 is at the operational pressure. When uninflated, flexible membrane 38 as shown in FIGS. 4G-H would appear as shown in FIGS. 4A-B.

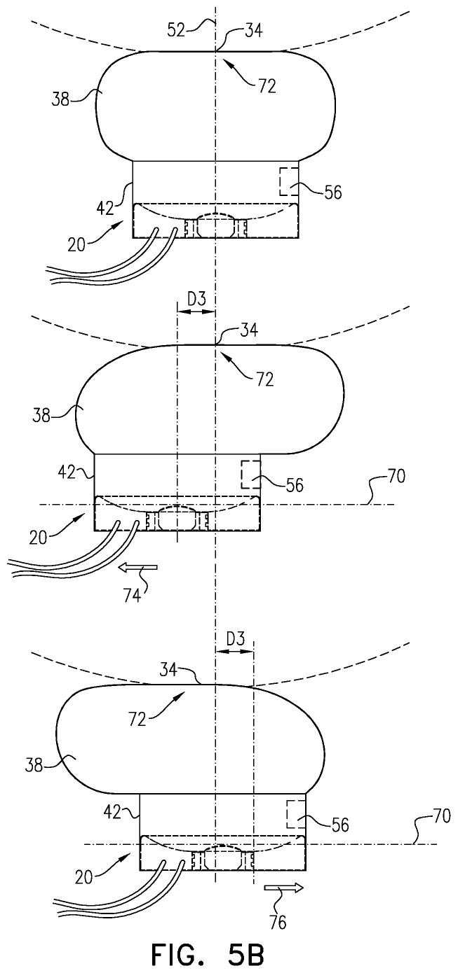

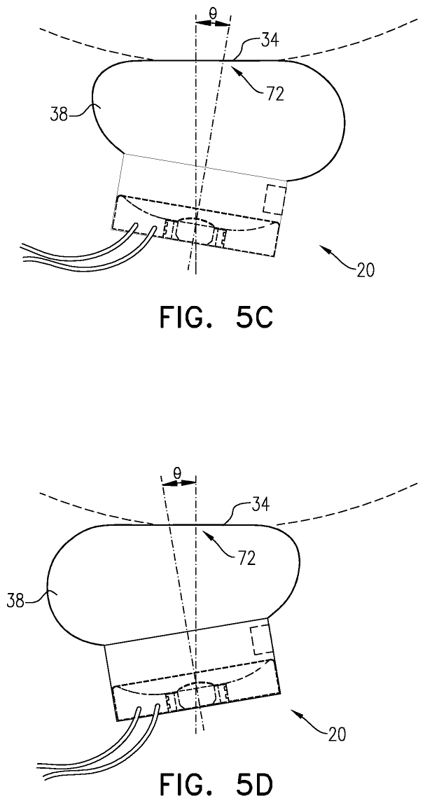

Reference is now made to FIGS. 5A-D, which are schematic illustrations of treatment head 20 and inflated flexible membrane 38 during motion of treatment head 20, in accordance with some applications of the present invention. As described hereinabove, the size and shape of flexible membrane 38 provides a liquid-filled space between treatment head 20 and perineum 34 of subject 25 when flexible membrane 38 is inflated with liquid, allowing treatment head 20 to be moved with 3-6 degrees of freedom relative to seat 32 of chair 28 while maintaining acoustic coupling with perineum 34 of subject 25. In order to accommodate for the movement of treatment head 20, during motion of treatment head 20 relative to seat 32 of chair 28, pressure regulator 56 maintains flexible membrane 38 pressed against perineum 34 of subject 25 by maintaining the pressure within internal cavity 44 at the operational pressure. Typically, pressure regulator 56 regulates the pressure within internal cavity 44 by regulating the volume of internal cavity 44 during motion of treatment head 20.

For example, FIG. 5A shows treatment head 20 translating along longitudinal axis 52 of treatment head 20. As treatment head 20 moves toward perineum 34 along longitudinal axis 52 (illustrated by upward arrow 58), pressure regulator 56 may reduce the volume of internal cavity 44 in order to prevent the pressure within internal cavity 44 increasing to above the operational pressure. Due to the flexibility of flexible membrane 38, the shape of flexible membrane 38 can change, allowing the liquid within internal cavity 44 to shift in order to accommodating the movement of treatment head 20 toward perineum 34. As treatment head moves away from perineum 34 along longitudinal axis 52 (illustrated by downward arrow pressure regulator 56 may increase the volume of internal cavity 44 in order to prevent the pressure within internal cavity 44 decreasing below the operational pressure.

For some applications, pressure regulator 56 includes a pressure sensor and a pump in fluid communication with the liquid within internal cavity 44. Housing 42 has at least one fluid port 62 in fluid communication with internal cavity 44 of housing 42. Pressure regulator 56 regulates the pressure within internal cavity 44 of housing 42 by regulating a volume of the liquid within internal cavity 44 of housing 42 using fluid port 62, e.g., by pumping the liquid in or out of fluid port 62. For some applications, housing 42 has two fluid ports 62, (a) a first fluid port 62 being a fluid inlet port 62a through which the liquid is received into internal cavity 44 of housing 42, and (b) the second fluid port 62, being a fluid outlet port 62b through which the liquid is drained from internal cavity 44 of housing 42. Pressure regulator 56 regulates the pressure within internal cavity 44 of housing 42 by regulating a volume of the liquid within internal cavity 44 of housing 42 using fluid inlet port 62a and fluid outlet port 62b.

Typically, the fluid port(s) are also used for circulating the liquid for the purposes of cooling, filtering, and degassing. In applications of the present invention where water permeable portion 46 of flexible membrane 38 is used, such that when flexible membrane 38 is pressed against perineum 34 of subject 25 liquid from within internal cavity 44 of housing 42 seeps through portion 46 of flexible membrane 38, an operator the system or practitioner may need to periodically verify that a sufficient amount of liquid remains within internal cavity 44 and to add liquid if needed.

With specific reference to FIG. 5A, typically, flexible membrane 38 and housing 42 of treatment head 20 are arranged such that when (a) subject 25 is sitting in chair 28 and (b) pressure regulator 56 is maintaining the pressure at the operational pressure during the motion of treatment head 20 relative to seat 32, treatment head 20 can translate along longitudinal axis 52 of treatment head 20 at least a distance D1 of 1 cm, e.g., at least 3 cm, away from perineum 34 of subject 25 without flexible membrane 38 losing contact with perineum 34 of subject 25. For some applications, treatment head 20 can translate along longitudinal axis 52 a distance D2 of 8 cm away from perineum 34 without flexible membrane 38 losing contact with perineum 34 of subject 25. For some applications, when subject 25 is sitting in chair 28, treatment head 20 is initially positioned at a zero-point along longitudinal axis 52 such that, when acoustically coupled to perineum 34 of subject 25, treatment head 20 can move 4 cm toward perineum 34 from the zero-point and 4 cm away from perineum 34 from the zero-point.

With specific reference to FIG. 5B, typically, flexible membrane 38 and housing 42 of treatment head 20 are arranged such that when (a) subject 25 is sitting in chair 28 and (b) pressure regulator 56 is maintaining the pressure within internal cavity 44 at the operational pressure during the motion of treatment head 20 relative to the seat 32, treatment head 20 can translate at least a distance D3 of 1 cm along an axis 70 that is perpendicular to longitudinal axis 52 of treatment head 20 (e.g., the x-axis and/or y-axis as shown hereinabove in FIG. 3A) without a contact portion 72 of flexible membrane 38 that is in contact with perineum 34 of subject 25 sliding with respect to perineum 34 of subject 25. Translation along perpendicular axis 70 is represented by left-pointing arrow 74 and right-pointing arrow 76 in FIG. 5B. For some applications, treatment head 20 can translate along perpendicular axis 70 a distance of 6 cm without contact portion 72 of flexible membrane 38 sliding with respect to perineum 34 of subject 25.

With specific reference to FIGS. 5C-D, typically, flexible membrane 38 and housing 42 of treatment head 20 are arranged such that when (a) subject 25 is sitting in chair 28 and (b) pressure regulator 56 is maintaining the pressure within internal cavity 44 at the operational pressure during the motion of treatment head 20 relative to seat 32, treatment head 20 can rotate by at least an angle θ (theta) of 5 degrees about an axis that is perpendicular to longitudinal axis 52 of treatment head 20 without contact portion 72 of flexible membrane 38 that is in contact with perineum 34 of subject 25 sliding with respect to perineum 34 of subject 25. Tilt angle θ (theta) is also shown in FIGS. 2C-D. Typically, treatment head 20 tilts by rotating about the x-axis and/or the y-axis as shown in FIG. 2B. For some applications, treatment head 20 can rotate by an angle θ (theta) of +/−30 degrees about an axis that is perpendicular to longitudinal axis 52 of treatment head 20 without contact portion 72 of flexible membrane 38 that is in contact with perineum 34 of subject 25 sliding with respect to perineum 34 of subject 25.

Reference is again made to FIG. 1. For some applications, control circuitry 36 controls the movement of treatment head 20 using a robotic arm 78. Treatment head 20 is coupled to a distal end 80 of robotic arm 78. Control circuitry 36 controls movement of treatment head 20 by controlling movement of robotic arm 78. For some applications, robotic arm 78 is coupled to chair-frame 30. Alternatively, robotic arm 78 may be separate from chair-frame 30. Movement of treatment head 20 using robotic arm 78 is the same as the movement of treatment head 20 described hereinabove with reference to FIGS. 2B-D, and FIGS. 5A-D, mutatis mutandis.

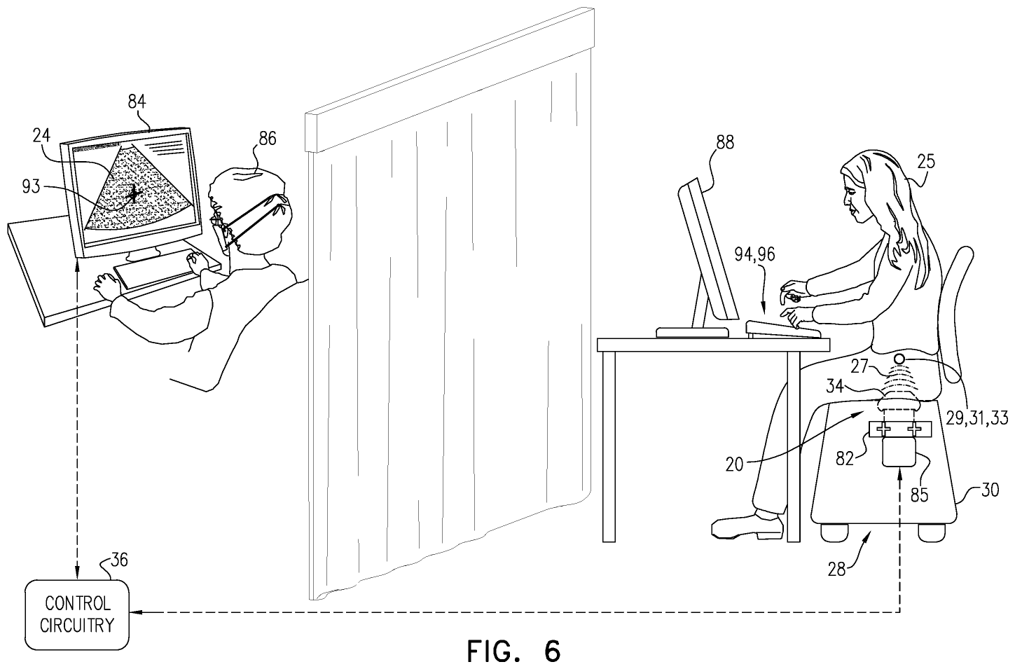

Reference is now made to FIG. 6, which is a schematic illustration of apparatus for pelvic floor diagnosis and treatment, in accordance with some applications of the present invention. For some applications, treatment head 20 may be disposed on a positioning platform 82 that is integrated into chair 28 and allows for treatment head 20 to be translated and rotated relative to the patient's body, as described hereinabove with reference to FIGS. 2B-D and FIGS. 5A-D, mutatis mutandis. An electromechanical unit 85 may incorporate gyroscopes to allow for the orientation and positioning of positioning platform 82.

Reference is now made to FIGS. 1 and 6. Typically, control circuitry 36 registers imaging acoustic field 27 and therapeutic acoustic field 29, such that imaging acoustic field 27 and therapeutic acoustic field 29 share a common coordinate system. Due to the registration of imaging acoustic field 27 and therapeutic acoustic field 29, control circuitry 36 is configured to show on a display 84 (a) sonogram 24 of the internal anatomy of subject 25 and (b) an image 93 of a focal region 33 of HIFU energy 31 with respect to the internal anatomy of subject 25 overlaid on sonogram 24 on display 84, such as is shown in FIGS. 1 and 6. Typically, display 84 is used by a practitioner performing the exam and/or treatment session. For some applications, a second display 88 is disposed such that second display 88 is visible to subject when subject 25 is sitting in chair 28. For some applications, second display 88 is an integrated part of chair 28, e.g., coupled to chair 28, such as is shown in FIG. 1. Alternatively, second display 88 is separate from chair 28, such as is shown in FIG. 6. Second display 88 may by disposed anywhere such that it is visible to subject 25 when subject 25 is sitting in chair 28, e.g., mounted to a wall in the vicinity of chair 28. For some applications, second display 88 may be a wearable display, e.g., virtual reality goggles, which subject 25 wears during the exam and/or treatment session.

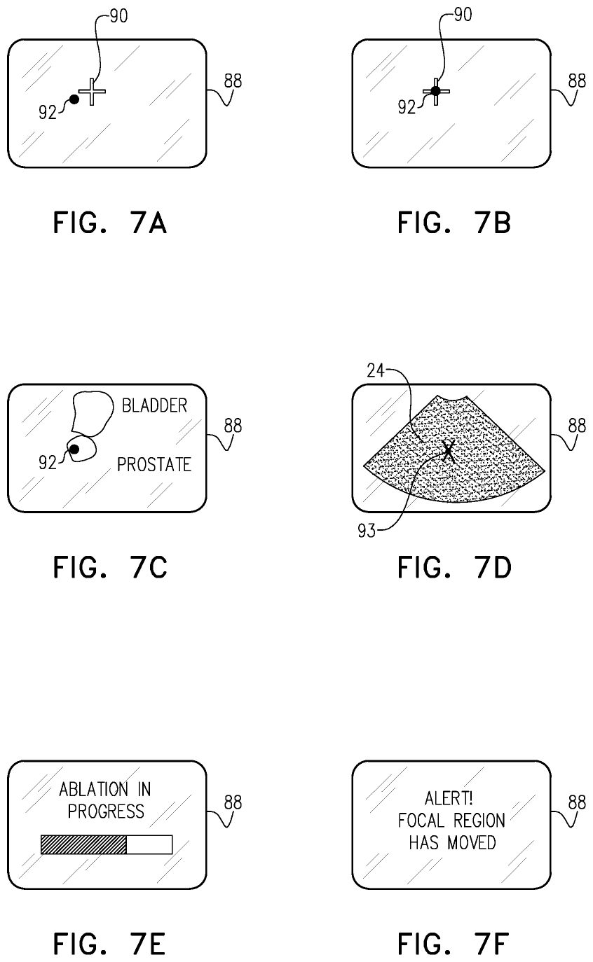

Reference is now made to FIGS. 7A-F, which are schematic illustrations of second display 88, in accordance with some applications of the present invention. Second display 88 allows subject 25 to be interactive in the exam and/or treatment session. Control circuitry 36 typically displays on second display 88 spatial information, based on (a) sonogram 24 of the internal anatomy of subject 25 and (b) focal region 33 of HIFU energy 31 with respect to the internal anatomy of subject 25. For some applications, control circuitry 36 displays the spatial information by displaying a spatial relationship between internal anatomy of subject 25 and focal region 33 of HIFU energy 31 with respect to the internal anatomy of subject 25. Some non-limiting examples of subject 25 being interactive in the exam and/or treatment session via second display 88 are as follows:

For some applications, subject 25 may be shown on display 88 a representation 90 of an intended anatomical target based on sonogram 24, and a representation 92 of a location focal region 33 of HIFU energy 31 with respect to the intended anatomical target, as illustrated in FIG. 7A. Subject 25 may then be asked to shift their position so as to align representation 90 of the intended anatomical target with representation 92 of focal region 33 of HIFU energy 31.

For some applications, when focal region 33 of HIFU energy 31 has been aligned with an intended anatomical target, subject 25 may be shown on second display 88 representation 90 of the intended anatomical target based on sonogram 24 with representation 92 of focal region 33 overlaid on the intended anatomical target, such as is shown in FIG. 7B. Being able to visualize focal region 33 and the intended anatomical target may help subject 25 minimize movement during ablation of the intended anatomical target, a process which can last, for example, a minute.

For some applications, subject 25 may be shown on second display 88 a labeled diagram of the internal anatomy based on sonogram 24 along with representation 92 of focal region 33 of HIFU energy 31 with respect to the internal anatomy, an example of which is shown in FIG. 7C.

For some applications, subject 25 may be shown on second display 88 (a) sonogram 24 of the internal anatomy of subject 25 and (b) image 93 of focal region 33 of HIFU energy 31 with respect to the internal anatomy of subject 25 overlaid on sonogram 24, i.e., the practitioner's display 84 and second display 88 may show the same thing, such as is shown in FIG. 7D.

For some applications, subject 25 may be shown on second display 88 an indication relating to a progression of a procedure that the subject is undergoing via treatment head 20. For example, such as is shown in FIG. 7E, subject 25 may be shown a status bar relating to an ablation so that they can visualize how much time remains—this may help them remain still and may also help them to endure discomfort that may be caused by HIFU energy 31.

For some applications, the indication relating to a progression of the procedure may be an alert shown to subject 25 on second display 88 if focal region 33 of HIFU energy 31 has moved with respect to internal anatomy of subject 25 due to movement of subject with respect to seat 32, an example of which is shown in FIG. 7F.

For some applications, control circuitry 36 includes user controls 94 (shown in FIGS. 1 and 6) configured to be used by subject 25 when subject 25 is sitting in chair 28 in order to provide feedback to practitioner 86 relating to a sensation that subject 25 is experiencing. For example, subject 25 can let practitioner 86 know if the treatment is causing them pain or discomfort, or a level of pain/discomfort. In cases where subject 25 may be asked to participate in the exam and/or treatment session by, for example, urinating, defecating, and/or ejaculating, subject 25 may indicate to practitioner 86 that he is ready to perform the requested biological function via user controls 94.

For some applications, such as is shown in FIG. 1, user controls 94 are integrated into chair 28. For example, user controls 94 may be buttons on arm rests 43 that subject 25 can press and/or second display 88 may be a touch screen with user controls 94 integrated into the software. For some applications, such as is shown in FIG. 6, user controls 94 are separate from chair 28. For example, display 88 may be a computer and user controls 94 may be a keyboard via which subject 25 communicates with practitioner 86. Display 88 and user controls 94 may be a conventional computer and keyboard, or a dedicated computer and keyboard that is sold commercially with chair 28. For some applications, user controls 94 include a stop-actuator 96 which subject 25 can actuate while sitting in chair 28. Stop-actuator 96 terminates the emission of HIFU energy 31 into subject 25. Thus, for example, if HIFU energy 31 starts to cause subject 25 pain, they can actuate the stop-actuator as an override in order to terminate the emission of HIFU energy 31. For some applications, user controls 94 may enable subject to control parameters of treatment head 20, such as for example, temperature control of the liquid within internal cavity 44.

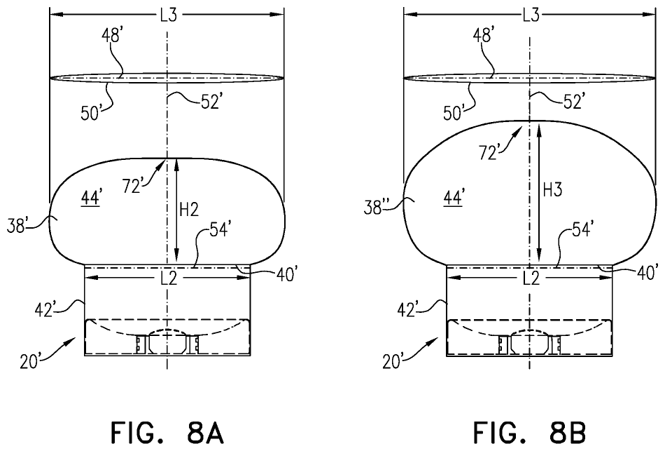

Reference is now made to FIGS. 8A-B, which are schematic illustrations of treatment head 20′, in accordance with some applications of the present invention. For some applications, alternatively to treatment head 20 which has fluid port(s) 62, treatment head 20′ may be commercially sold without the fluid ports(s) 62 as described hereinabove. Internal cavity 44′ formed by a flexible membrane 38′ and a housing 42′ is pre-filled with a fixed volume of degassed liquid, e.g., degassed oil or water, such that ultrasound imaging probe 22 and HIFU transducer 26 are in direct contact with the liquid. Flexible membrane 38′ is inflated outwards from perimeter 40′ of housing 42′ due to pressure from the liquid within internal cavity 44′, and when subject 25 is sitting in chair 28 perineum 34 of subject 25 is acoustically coupled to treatment head 20′ via flexible membrane 38′, flexible membrane 38′ being pressed against perineum 34 of subject 25 due to the pressure. Typically, pressure within internal cavity 44′ of treatment head 20′ is at least 1.2 atm and/or less than 2 atm. Typically, flexible membrane 38′ is coupled to housing 42′ such that when subject 25 is not sitting in chair 28, an uncompressed height H2 of inflated flexible membrane 38′ along longitudinal axis 52′ of treatment head 20′ is at least 2 cm and/or less than 4 cm, as illustrated in FIG. 8A. For some applications, in addition to be flexible, flexible membrane 38′ may also be elastic. In this case of flexible membrane 38′ being an elastic membrane 38″, elastic membrane 38″ is coupled to housing 42′ such that when subject 25 is not sitting in chair 28, an uncompressed height H3 of inflated elastic membrane 38″ along longitudinal axis 52′ of treatment head 20′ is at least 2 cm and/or less than 12 cm, as illustrated in FIG. 8B.

Typically, flexible membrane 38′ and elastic membrane 38″ are sized and shaped such that a major axis 48′ of a projection 50′, taken along longitudinal axis 52′ of treatment head of inflated flexible membrane 38′ and/or of inflated elastic membrane 38″ has a length L3 that is at least 6 cm and/or less than 12 cm. Typically, length L3 of major axis 48′ is (a) at least 2 cm and/or less than 8 cm longer, and/or (b) at least 20% and/or less than 100% larger than a major axis 54′ of perimeter 40′ of housing 42′, as illustrated in FIGS. 8A-D. Major axis 54′ of perimeter 40′ of housing 42′ typically has a length L2 that is at least 4 cm and/or less than 12 cm.

Unlike treatment head 20, treatment head 20′ does not have a pressure regulator to regulate the pressure within the internal cavity by regulating the volume of liquid. Therefore, when subject 25 is sitting in chair 28, during motion of treatment head 20′ relative to seat 32 control circuitry 36 maintains treatment head 20′ in close enough proximity to perineum 34 of subject 25 so as maintain the flexible membrane pressed against the perineum of the subject and regulates the pressure within internal cavity 44′ by regulating a force with which flexible membrane 38′ or elastic membrane 38″ is pressed against perineum 34.

Typically, flexible membrane 38′ or elastic membrane 38″ and housing 42′ of treatment head 20′ are arranged such that when (a) subject 25 is sitting in chair 28 and (b) control circuitry is maintaining flexible membrane 38′ or elastic membrane 38″ pressed against perineum 34 of subject 25 during motion of treatment head 20 relative to seat 32, treatment head 20′ can translate at least 1 cm along an axis that is perpendicular to longitudinal axis 52′ of treatment head 20 without a contact portion 72′ of flexible membrane 38′ or elastic membrane 38″ that is in contact with perineum 34 of subject 25 sliding with respect to perineum 34 of subject 25. This translation along an axis that is perpendicular to longitudinal axis 52′ is the same as the translational motion of treatment head 20 along axis 70 as described with reference to FIG. 5B, mutatis mutandis. For some applications, treatment head 20′ can translate along an axis that is perpendicular to longitudinal axis 52′ a distance of 6 cm without contact portion 72′ of flexible membrane 38′ or elastic membrane 38″ sliding with respect to perineum 34 of subject 25.

Typically, flexible membrane 38′ or elastic membrane 38″ and housing 42′ of treatment head 20′ are arranged such that when (a) subject 25 is sitting in chair 28 and (b) control circuitry 36 is maintaining flexible membrane 38′ or elastic membrane 38″ pressed against perineum 34 of subject 25 during motion of treatment head 20′ relative to seat 32, treatment head 20′ can rotate by at least an angle θ (theta) of 5 degrees about an axis that is perpendicular to longitudinal axis 52′ of treatment head 20′ without contact portion 72′ of flexible membrane 38′ or elastic membrane 38″ that is in contact with perineum 34 of subject sliding with respect to perineum 34 of subject 25. This rotation about an axis that is perpendicular to longitudinal axis 52′ is the same as the rotational motion of treatment head 20 described with reference to FIGS. 2B-D and FIGS. 5C-D, mutatis mutandis For some applications, treatment head 20′ can rotate by an angle θ (theta) of +/−30 degrees about an axis that is perpendicular to longitudinal axis 52′ of treatment head 20′ without contact portion 72′ of flexible membrane 38′ or elastic membrane 38″ that is in contact with perineum 34 of subject 25 sliding with respect to perineum 34 of subject 25.

Internal cavity 44′ of housing 42′ is typically filled with degassed liquid, which is incompressible. In the case of elastic membrane 38″ being sealably coupled to perimeter 40′ of housing 42′ when (a) subject 25 is sitting in chair 28 and (b) control circuitry 36 is maintaining elastic membrane 38″ pressed against perineum 34 of subject 25 during motion of treatment head 20′ relative to seat 32, treatment head 20′ can translate along longitudinal axis 52′ of treatment head 20′ at least 1 cm, e.g., at least 3 cm, away from perineum 34 of subject 25 without elastic membrane 38″ losing contact with perineum 34 of subject 25. The elasticity of elastic membrane 38″ enables the liquid within internal cavity 44′ to shift even while inflated elastic membrane 38″ is being pressed against perineum 34, thus enabling this translational movement along longitudinal axis 52′. This translation along longitudinal axis 52′ is the same as the translational motion of treatment head 20 along longitudinal axis 52 as described with reference to FIG. 5A, mutatis mutandis.

The present application may be used in combination with methods for distinguishing tissues (e.g., for cancer detection) as disclosed in PCT/IL2021/051120 to Ben-Ezra, entitled “Ultrasound tissue differentiation system,” which claims the priority of US 63/079,485. Each of these applications is incorporated herein by reference. Four methods of ultrasound-based cancer detection are disclosed: 1. Regular ultrasound is used for detection of cancers having different acoustic response than surrounding normal tissue; 2. Acoustic radiation force imaging may be used to find the elasticity of tissue at low frequencies; 3. HIFU imaging may be used to determine acoustic impedance of tissue at acoustic (e.g., ultrasonic) frequencies and/or tissue response to higher HIFU harmonics; and 4. focused ultrasound may be used to heat tissue, and related imaging methods may be used to measure resulting temperature changes, allowing for determination of the heating response of tissue, which will in general be different for cancerous tissue as compared to noncancerous tissue. The four methods above are combined after suitable registration to provide a clearer image of cancerous vs. noncancerous tissue.

PCT/IL2021/051120 to Ben-Ezra further describes assessing a characteristic of a tissue. A set of one or more acoustic transducers transmits a first acoustic field at a first frequency into the tissue, generating oscillatory motion at the first frequency of scatterers disposed in the tissue. A second acoustic field at a second frequency higher than the first frequency is transmitted into the tissue. Echo data is received due to the second acoustic field scattering off an oscillating scatterer that is oscillating at the first frequency. A computer processor derives an indication of acoustic impedance of the tissue based on the echo data, and drives an output device to output an indication of whether the tissue is or may be a tumor, based on the indication of the acoustic impedance.

Applications of the invention described herein can take the form of a computer program product accessible from a computer-usable or computer-readable medium (e.g., a non-transitory computer-readable medium) providing program code for use by or in connection with a computer or any instruction execution system, such as control circuitry 36. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD. For some applications, cloud storage, and/or storage in a remote server is used.

A data processing system suitable for storing and/or executing program code will include at least one processor (e.g., control circuitry 36) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the methods described herein can be implemented by computer program instructions. These computer program instructions may be provided to a processor of a general-purpose computer, special purpose computer, or other programmable data processing apparatus to produce a machine, such that the instructions, which execute via the processor of the computer (e.g., control circuitry 36) or other programmable data processing apparatus, create means for implementing the functions/acts specified in the methods described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the methods described in the present application. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the methods described in the present application.

Control circuitry 36 is typically a hardware device programmed with computer program instructions to produce a special purpose computer. For example, when programmed to perform the methods described herein, the computer processor typically acts as a special purpose computer processor. Typically, the operations described herein that are performed by computer processors transform the physical state of a memory, which is a real physical article, to have a different magnetic polarity, electrical charge, or the like depending on the technology of the memory that is used.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with a subject, the apparatus comprising:
a treatment head comprising:
an ultrasound imaging probe configured to generate a sonogram of internal anatomy of the subject by generating an imaging acoustic field; and
a high intensity focused ultrasound (HIFU) transducer configured to generate a therapeutic acoustic field by emitting HIFU energy into the subject;
a chair comprising a chair-frame and a seat, wherein the chair-frame, the seat, and the treatment head are arranged such that when the subject is sitting in the chair a perineum of the subject is acoustically coupled to the treatment head; and
control circuitry configured to (a) control movement of the treatment head relative to the seat, (b) operate the ultrasound imaging probe to generate at least one sonogram of internal anatomy of the subject through the perineum of the subject, and (c) operate the HIFU transducer to emit HIFU energy into the body of the subject through the perineum of the subject.

2. The apparatus according to claim 1, wherein the treatment head is moveably coupled to the chair-frame.

3. The apparatus according to any one of claims 1-2, wherein the HIFU transducer and the ultrasound imaging probe are fixed to the treatment head such that movement of the treatment head relative to the seat moves the HIFU transducer and the ultrasound imaging probe relative to the seat.

4. The apparatus according to claim 3, wherein the control circuitry is configured to control translation of the treatment head along a longitudinal axis of the treatment head, and along an axis that is perpendicular to the longitudinal axis.

5. The apparatus according to claim 3, wherein the control circuitry is configured to control rotation of the treatment head about an axis that is perpendicular to a longitudinal axis of the treatment head.

6. The apparatus according to claim 3, wherein the control circuitry is configured to control rotation of the treatment head about a longitudinal axis of the treatment head.

7. The apparatus according to claim 3, wherein the ultrasound imaging probe is configured to rotate relative to the treatment head about a longitudinal axis of the treatment head, and wherein the control circuitry is further configured to control the rotation of the ultrasound imaging probe relative to the treatment head.

8. The apparatus according to claim 7, wherein the HIFU transducer and the ultrasound imaging probe are coaxial, and the ultrasound imaging probe is configured to rotate within a central bore of the HIFU transducer.

9. The apparatus according to any one of claims 1-2, wherein the treatment head comprises:
a housing, in which the ultrasound imaging probe and the HIFU transducer are disposed; and
a flexible membrane sealably coupled to a perimeter of the housing, the flexible membrane and the housing forming an internal cavity that is configured to be filled with a liquid such that, when the internal cavity of the housing is filled with the liquid the ultrasound imaging probe and the HIFU transducer are in direct contact with the liquid, wherein:
the flexible membrane is configured to inflate outwards from the perimeter of the housing due to pressure from the liquid within the internal cavity, and
when the subject is sitting in the chair the perineum of the subject is acoustically coupled to the treatment head via the flexible membrane, the flexible membrane being pressed against the perineum of the subject due to the pressure.

10. The apparatus according to claim 9, wherein the liquid is degassed water.

11. The apparatus according to claim 9, wherein at least a portion of the flexible membrane is water permeable and is configured such that when the flexible membrane is pressed against the perineum of the subject, liquid from within the internal cavity of the housing seeps through the at least a portion of the flexible membrane such that the perineum is acoustically coupled to the flexible membrane via the liquid that seeped through the at least a portion of the flexible membrane.

12. The apparatus according to claim 9, wherein the flexible membrane is arranged such that a major axis of a projection of the uninflated flexible membrane taken along a longitudinal axis of the treatment head of is 6-12 cm.

13. The apparatus according to claim 12, wherein the flexible membrane is arranged such that the major axis of the projection of the uninflated flexible membrane taken along the longitudinal axis of the treatment head is 2-8 cm longer than a major axis of the perimeter of the housing to which the flexible membrane is sealably coupled.

14. The apparatus according to claim 12, wherein the flexible membrane is arranged such that the major axis of the projection of the uninflated flexible membrane taken along the longitudinal axis of the treatment head is 20-100% larger than a major axis of the perimeter of the housing to which the flexible membrane is sealably coupled.

15. The apparatus according to claim 9, further comprising a pressure regulator coupled to the housing and configured to regulate the pressure within the internal cavity of the housing.

16. The apparatus according to claim 15, wherein:
the housing comprises a fluid port in fluid communication with the internal cavity of the housing, and
the pressure regulator is configured to regulate the pressure within the internal cavity of the housing by regulating a volume of the liquid within the internal cavity of the housing using the fluid port.

17. The apparatus according to claim 16, wherein:
the fluid port is a first fluid port and the housing further comprises a second fluid port, (a) the first fluid port being a fluid inlet port through which the liquid is received into the internal cavity of the housing, (b) the second fluid port being a fluid outlet port through which the liquid is drained from the internal cavity of the housing, and
the pressure regulator is configured to regulate the pressure within the internal cavity of the housing by regulating a volume of the liquid within the internal cavity of the housing using the fluid inlet port and the fluid outlet port.

18. The apparatus according to claim 16, wherein the flexible membrane is coupled to the housing such that (a) when the flexible membrane is inflated by the internal cavity of the housing being filled with a volume of liquid such that the pressure within the internal cavity is 1.2 atm, and (b) the subject is not sitting in the chair, an uncompressed height of the inflated flexible membrane along a longitudinal axis of the treatment head is 2-12 cm.

19. The apparatus according to claim 18, wherein the pressure regulator is configured such that, when the subject is sitting in the chair, the pressure regulator maintains the flexible membrane pressed against the perineum of the subject by maintaining the pressure within the internal cavity at an operational pressure.

20. The apparatus according to claim 19, wherein the operational pressure is 1.2-2 atm.

21. The apparatus according to claim 19, wherein the pressure regulator is configured such that, when the subject is sitting in the chair, during motion of the treatment head relative to the seat, the pressure regulator maintains the flexible membrane pressed against the perineum of the subject by maintaining the pressure within the internal cavity at the operational pressure.

22. The apparatus according to claim 21, wherein the flexible membrane and the housing of the treatment head are arranged such that when (a) the subject is sitting in the chair and (b) the pressure regulator is maintaining the pressure at the operational pressure during the motion of the treatment head relative to the seat, the treatment head can translate along the longitudinal axis of the treatment head at least 1 cm away from the perineum of the subject without the flexible membrane losing contact with the perineum of the subject.

23. The apparatus according to claim 21, wherein the flexible membrane and the housing of the treatment head are arranged such that when (a) the subject is sitting in the chair and (b) the pressure regulator is maintaining the pressure at the operational pressure during the motion of the treatment head relative to the seat, the treatment head can translate at least 1 cm along an axis that is perpendicular to the longitudinal axis of the treatment head without a contact portion of the flexible membrane that is in contact with the perineum of the subject sliding with respect to the perineum of the subject.

24. The apparatus according to claim 21, wherein the flexible membrane and the housing of the treatment head are arranged such that when (a) the subject is sitting in the chair and (b) the pressure regulator is maintaining the pressure at the operational pressure during the motion of the treatment head relative to the seat, the treatment head can rotate by at least 5 degrees about an axis that is perpendicular to the longitudinal axis of the treatment head without a contact portion of the flexible membrane that is in contact with the perineum of the subject sliding with respect to the perineum of the subject.

25. The apparatus according to any one of claims 1-2, further comprising a robotic arm, the treatment head being coupled to a distal end of the robotic arm, wherein the control circuitry is configured to control movement of the treatment head relative to the seat by controlling movement of the robotic arm.

26. The apparatus according to claim 25, wherein a proximal end of the robotic arm is coupled to the chair-frame.

27. The apparatus according to claim 25, wherein the robotic arm is configured to move the treatment head in a plurality of degrees of freedom.

28. The apparatus according to claim 27, wherein the HIFU transducer and the ultrasound imaging probe are fixed to the treatment head such that movement of the treatment head relative to the seat moves the HIFU transducer and the ultrasound imaging probe relative to the seat.

29. The apparatus according to claim 28, wherein the robotic arm is configured to translate the treatment head along a longitudinal axis of the treatment head, and along an axis that is perpendicular to the longitudinal axis.

30. The apparatus according to claim 28, wherein the robotic arm is configured to rotate the treatment head about an axis that is perpendicular to a longitudinal axis of the treatment head.

31. The apparatus according to claim 28, wherein the robotic arm is configured to rotate the treatment head about a longitudinal axis of the treatment head.

32. The apparatus according to claim 28, wherein the ultrasound imaging probe is configured to rotate relative to the treatment head about a longitudinal axis of the treatment head, and wherein the robotic arm is further configured to control the rotation of the ultrasound imaging probe relative to the treatment head.

33. The apparatus according to claim 32, wherein the HIFU transducer and the ultrasound imaging probe are coaxial, and the ultrasound imaging probe is configured to rotate within a central bore of the HIFU transducer.

34. The apparatus according to any one of claims 1-2, wherein the control circuitry is configured to register the imaging acoustic field and the therapeutic acoustic field, such that the imaging acoustic field and the therapeutic acoustic field share a common coordinate system.

35. The apparatus according to claim 34, wherein the apparatus is for use with a display and, due to the registration of the imaging acoustic field and the therapeutic acoustic field, the control circuitry is configured to show on the display (a) the sonogram of the internal anatomy of the subject and (b) a focal region of the HIFU energy with respect to the internal anatomy of the subject overlaid on the sonogram of the internal anatomy of the subject on the display.

36. The apparatus according to claim 35, wherein the display is a first display configured to be used by a practitioner, the apparatus is further for use with a second display disposed such that the second display is visible to the subject when the subject is sitting in the chair, and the control circuitry is configured to display on the second display spatial information based on (a) the sonogram of the internal anatomy of the subject and (b) a focal region of the HIFU energy with respect to the internal anatomy of the subject.

37. The apparatus according to claim 36, wherein the control circuitry is configured to display the spatial information by displaying a spatial relationship between internal anatomy of the subject and the focal region of the HIFU energy with respect to the internal anatomy of the subject.

38. The apparatus according to claim 36, wherein the control circuitry is configured to display (a) the sonogram of the internal anatomy of the subject and (b) a focal region of the HIFU energy with respect to the internal anatomy of the subject overlaid on the sonogram of the internal anatomy of the subject.

39. The apparatus according to claim 36, wherein the second display is coupled to the chair-frame.

40. The apparatus according to claim 36, wherein the control circuitry is configured to display an indication to the subject relating to a progression of a procedure that the subject is undergoing, the procedure being performed via the treatment head.

41. The apparatus according to claim 40, wherein the control circuitry is configured to display the indication by displaying an alert to the subject on the second display if the focal region of the HIFU energy has moved with respect to internal anatomy of the subject due to movement of the subject with respect to the seat.

42. The apparatus according to any one of claims 1-2, wherein the control circuitry comprises user controls configured to be used by the subject when the subject is sitting in the chair in order to provide feedback to the practitioner relating to a sensation that the subject is experiencing.

43. The apparatus according to claim 42, wherein the user controls comprise a stop-actuator which the subject can actuate while sitting in the chair, the stop-actuator being configured to terminate the emission of HIFU energy into the subject.

44. The apparatus according to any one of claims 1-2, wherein the treatment head comprises:
a housing, in which the ultrasound imaging probe and the HIFU transducer are disposed; and
a flexible membrane sealably coupled to a perimeter of the housing, the flexible membrane and the housing forming an internal cavity that is filled with a volume of liquid such that the ultrasound imaging probe and the HIFU transducer are in direct contact with the liquid, wherein:
the flexible membrane is inflated outwards from the perimeter of the housing due to pressure from the liquid within the internal cavity, and
when the subject is sitting in the chair the perineum of the subject is acoustically coupled to the treatment head via the flexible membrane, the flexible membrane being pressed against the perineum of the subject due to the pressure.

45. The apparatus according to claim 44, wherein at least a portion of the flexible membrane is water permeable and is configured such that when the flexible membrane is pressed against the perineum of the subject, liquid from within the internal cavity of the housing seeps through the at least a portion of the flexible membrane such that the perineum is acoustically coupled to the flexible membrane via the liquid that seeped through the at least a portion of the flexible membrane.

46. The apparatus according to claim 44, wherein the flexible membrane is coupled to the housing such that the pressure within the internal cavity is 1.2-2 atm, and is configured such that when the subject is not sitting in the chair, an uncompressed height of the inflated flexible membrane along a longitudinal axis of the treatment head is 2-4 cm.

47. The apparatus according to claim 46, wherein the flexible membrane is arranged such that a major axis of a projection of the inflated flexible membrane taken along a longitudinal axis of the treatment head when the subject is not sitting in the chair of is 6-12 cm.

48. The apparatus according to claim 47, wherein the flexible membrane is arranged such that the major axis of the projection of the inflated flexible membrane taken along the longitudinal axis of the treatment head when the subject is not sitting in the chair is 2-8 cm longer than a major axis of the perimeter of the housing to which the flexible membrane is sealably coupled.

49. The apparatus according to claim 47, wherein the flexible membrane is arranged such that the major axis of the projection of the inflated flexible membrane taken along the longitudinal axis of the treatment head when the subject is not sitting in the chair is 20-100% larger than a major axis of the perimeter of the housing to which the flexible membrane is sealably coupled.

50. The apparatus according to claim 46, wherein the control circuitry is configured such that, when the subject is sitting in the chair, during motion of the treatment head relative to the seat, the control circuitry maintains the flexible membrane pressed against the perineum of the subject.

51. The apparatus according to claim 50, wherein the control circuitry is configured such that, when the subject is sitting in the chair, during motion of the treatment head relative to the seat, the control circuitry regulates the pressure within the internal cavity by regulating a force with which the flexible membrane is pressed against the perineum.

52. The apparatus according to claim 50, wherein the flexible membrane and the housing of the treatment head are arranged such that when (a) the subject is sitting in the chair and (b) the control circuitry is maintaining the flexible membrane pressed against the perineum of the subject during motion of the treatment head relative to the seat, the treatment head can translate at least 1 cm along an axis that is perpendicular to the longitudinal axis of the treatment head without a contact portion of the flexible membrane that is in contact with the perineum of the subject sliding with respect to the perineum of the subject.

53. The apparatus according to claim 50, wherein the flexible membrane and the housing of the treatment head are arranged such that when (a) the subject is sitting in the chair and (b) the control circuitry is maintaining the flexible membrane pressed against the perineum of the subject during motion of the treatment head relative to the seat, the treatment head can rotate by at least 5 degrees about an axis that is perpendicular to the longitudinal axis of the treatment head without a contact portion of the flexible membrane that is in contact with the perineum of the subject sliding with respect to the perineum of the subject.

54. The apparatus according to claim 44, wherein the flexible membrane is an elastic membrane, and wherein the elastic membrane is (a) coupled to the housing such that the pressure within the internal cavity is 1.2-2 atm, and (b) configured such that when the subject is not sitting in the chair, an uncompressed height of the inflated elastic membrane along a longitudinal axis of the treatment head is 2-12 cm.

55. The apparatus according to claim 54, wherein the elastic membrane is arranged such that a major axis of a projection of the inflated elastic membrane taken along a longitudinal axis of the treatment head when the subject is not sitting in the chair of is 6-12 cm.

56. The apparatus according to claim 55, wherein the elastic membrane is arranged such that the major axis of the projection of the inflated elastic membrane taken along the longitudinal axis of the treatment head when the subject is not sitting in the chair is 2-8 cm longer than a major axis of the perimeter of the housing to which the elastic membrane is sealably coupled.

57. The apparatus according to claim 55, wherein the elastic membrane is arranged such that the major axis of the projection of the inflated elastic membrane taken along the longitudinal axis of the treatment head when the subject is not sitting in the chair is 20-100% larger than a major axis of the perimeter of the housing to which the elastic membrane is sealably coupled.

58. The apparatus according to claim 54, wherein the control circuitry is configured such that, when the subject is sitting in the chair, during motion of the treatment head relative to the seat, the control circuitry maintains the elastic membrane pressed against the perineum of the subject.

59. The apparatus according to claim 58, wherein the control circuitry is configured such that, when the subject is sitting in the chair, during motion of the treatment head relative to the seat, the control circuitry regulates the pressure within the internal cavity by regulating a force with which the elastic membrane is pressed against the perineum.

60. The apparatus according to claim 58, wherein the elastic membrane and the housing of the treatment head are arranged such that when (a) the subject is sitting in the chair and (b) the control circuitry is maintaining the elastic membrane pressed against the perineum of the subject during motion of the treatment head relative to the seat, the treatment head can translate along the longitudinal axis of the treatment head at least 1 cm away from the perineum of the subject without the elastic membrane losing contact with the perineum of the subject.

61. The apparatus according to claim 58, wherein the elastic membrane and the housing of the treatment head are arranged such that when (a) the subject is sitting in the chair and (b) the control circuitry is maintaining the elastic membrane pressed against the perineum of the subject during motion of the treatment head relative to the seat, the treatment head can translate at least 1 cm along an axis that is perpendicular to the longitudinal axis of the treatment head without a contact portion of the elastic membrane that is in contact with the perineum of the subject sliding with respect to the perineum of the subject.

62. The apparatus according to claim 58, wherein the elastic membrane and the housing of the treatment head are arranged such that when (a) the subject is sitting in the chair and (b) the control circuitry is maintaining the elastic membrane pressed against the perineum of the subject during motion of the treatment head relative to the seat, the treatment head can rotate by at least 5 degrees about an axis that is perpendicular to the longitudinal axis of the treatment head without a contact portion of the elastic membrane that is in contact with the perineum of the subject sliding with respect to the perineum of the subject.
